(19) United States
(12) Patent Application Publication
(10) Pub. No.: US 2006/0257449 A1
(43) Pub. Date: Nov. 16, 2006

(54) Methods, compositions, systems, and devices for bone fusion

(76) Inventors: Didier Billy, Maastricht (NL); Frank Everaerts, Maastricht (NL); Michel Verhoeven, Maastricht (NL)

Correspondence Address: MUETING, RAASCH & GEBHARDT, P.A., P.O. BOX 581415, MINNEAPOLIS, MN, 55458, US

(21) Appl. No.: 11/129,977

(22) Filed: May 16, 2005

(51) Int. Cl. A61K 35/12; A61K 38/18

(52) U.S. Cl. 424/426; 514/12; 424/93.7

(57) ABSTRACT

The present invention is directed to methods, compositions, systems, and medical devices for fusing bone.

### BACKGROUND

When conservative treatment has failed, lumbar spinal fusion has been accepted as an option for patients with severe discogenic pain from instability and lumbar degenerative pathologies. The ultimate goal of fusion is the elimination of movement between the motion segments that will reduce or abolish the pain.

Spinal fusion is the uniting of two or more motion segments (disc space and paired facet joint, i.e., a single motion segment) together by the placement of bone graft. This fusion process is not only the immediate result of placement of a cage bone graft connecting the two motion segments but also the result of the body's healing process resulting in the formation of new bone material. Therefore, new approaches in lumbar fusion surgery attempt to enhance the body's healing potential to promote this fusion process.

Currently, the widely accepted surgical method of lumbar interbody fusion is generally performed with nonresorbable interbody fusion cages filled with autologous bone. The iliac crest remains the most readily available source of autologous bone, but the harvesting procedure is associated with a marked increase in morbidity. Reported major complications include iliac wing fracture and/or instability, as well as vascular tears and/or hematoma requiring surgical revision and severe pain.

Recent developments and knowledge in the field of tissue engineering offer opportunities for the development of new alternatives for bone graft materials that provide a fusion outcome equal, or superior, to that of autologous bone. Although autologous bone grafts contain both marrow cell elements and osteogenic cells, the fusion is a complex biological process requiring adequate blood supply and local growth factor accumulation. Therefore, an ideal biocompatible graft substitute should have osteoconductive and osteoinductive properties with an acceptable mechanical strength.

Because of the limitation in using autologous bone graft many synthetic materials exist as an alternative. Those various materials include hydroxyapatite (HA), tricalcium phosphate (TCP), biphasic calcium phosphate (BCP), collagen, and demineralized bone matrix. Moreover, bioresorbable cages made of polylactic acid with an elasticity modulus resembling that of vertebral bone could be used as a temporary carrier for synthetic filling material.

Posterolateral spine fusion is a very challenging area for bone formation/regeneration. Osteoconductive bone graft materials do not usually perform well in such an environment. Thus, compositions and systems for bone fusion are still needed.

### SUMMARY

The present invention is directed to methods, compositions, systems, and medical devices for fusing bone, particularly fusing vertebrae within the spine of a subject. The bone-fusion composition includes a matrix for bone formation and a growth factor protector and potentiator. Such compositions can be used in systems and medical devices that include cage devices for fusing vertebrae, for example.

The matrix for bone formation preferably includes an osteoconductive carrier such as a calcium phosphate, particularly biphasic calcium phosphate, although other matrices can be used including, for example, collagen, alginate, or combinations thereof.

The growth factor protector and potentiator is typically a heparin-binding growth factor protector and potentiator (preferably, a dextran derivative). The growth factor protector and potentiator is preferably selected from the polymers described in U.S. Pat. App. Pub. Nos. 2001/0021758 or 2001/0023246, or U.S. Pat. No. 6,689,741.

The bone-fusion composition can also be used in conjunction with a cage device (e.g., an interbody fusion cage), which can be made of a resorbable or nonresorbable material.

In one embodiment, the present invention provides a bone-fusion system that includes a bone-fusion composition, wherein the bone-fusion composition includes biphasic calcium phosphate, and a polymer having the general formula (I):
AₐXₓY_{y}
wherein:

A represents a monomer which is substituted with independently selected X and Y groups;

X represents a carboxyl group bonded to monomer A and is contained within a group according to the following formula: —R—COO—R′, in which R is a bond or an aliphatic hydrocarbon chain, optionally branched and/or unsaturated, and which can contain one or more aromatic rings except for benzylamine and benzylamine sulfonate, and R′ represents a hydrogen atom, Y, or a cation;

Y represents a sulfate of sulfonate group bonded to a monomer A and is contained within a group according to one of the following formulas: —R—O—SO₃—R″, —R—N—SO₃—R″, —R—SO₃—R″, in which R is a bond or an aliphatic hydrocarbon chain, optionally branched and/or unsaturated, and which can contain one or more aromatic rings except for benzylamine and benzylamine sulfonate, and R″ represents a hydrogen atom or a cation;

a represents the number of the monomer A such that the mass of said polymers of formula (I) is greater than 5,000 daltons;

x represents a substitution rate of the monomer A by the groups X, which is 20% to 150%; and

y represents a substitution rate of the monomers A by the groups Y, which is 30% to 150%.

In another embodiment, the present invention provides a bone-fusion system that includes a bone-fusion composition, wherein the bone-fusion composition includes biphasic calcium phosphate and a polymer having the general formula (II):
AₐXₓY_{y}Z_{z}
wherein:

A represents a monomer based on glucose which is substituted with independently selected X, Y, and Z groups;

X represents a carboxyl group bonded to monomer A and is contained within a group according to the following formula: —R—COO—R′, in which R is a bond or an aliphatic hydrocarbon chain, optionally branched and/or unsaturated, and which can

contain one or more aromatic rings except for benzylamine and benzylamine sulfonate, and R′ represents a hydrogen atom, Y, Z, or a cation;

Y represents a sulfate of sulfonate group bonded to a monomer A and is contained within a group according to one of the following formulas: —R—O—SO₃—R″, —R—N—SO₃—R″, —R—SO₃—R″, in which R is a bond or an aliphatic hydrocarbon chain, optionally branched and/or unsaturated, and which can contain one or more aromatic rings except for benzylamine and benzylamine sulfonate, and R″ represents a hydrogen atom, Z, or a cation;

Z is selected from the group consisting of amino acids, fatty acids, fatty alcohols, ceramides, or derivatives thereof, and nucleotide addressing sequences;

a represents the number of the monomer A such that the mass of said polymers of formula (II) is greater than 5,000 daltons;

x represents a substitution rate of the monomer A by the groups X, which is 20% to 150%;

y represents a substitution rate of the monomer A by the groups Y, which is 30% to 150%; and

z represents the rate of substitution of the monomer A by groups Z, which is 0 to 50%.

The present invention also provides methods and medical devices that include the bone-fusion systems and compositions described herein.

In one embodiment, a method of fusing bone is provided that includes: providing a bone-fusion system of the present invention that includes a bone-fusion composition; placing the composition in contact with bone to be fused; and allowing the bone-fusion composition to harden and fuse the bone.

In another embodiment, a method of fusing bone is provided that includes: providing a bone-fusion system that includes a bone-fusion composition, wherein the bone-fusion composition includes: a growth factor protector and potentiator; and a matrix for bone formation; placing the composition in contact with bone to be fused; and allowing the bone-fusion composition to harden and fuse the bone.

In one embodiment, a medical device is provided that includes a cage device and a bone-fusion composition, wherein the bone-fusion composition includes: a growth factor protector and potentiator; and a matrix for bone formation.

Herein, “bone fusion” refers to permanently joining bone in order to prevent motion, particularly between vertebrae. Spinal fusion is the permanent joining of two or more motion segments (disc space and paired facet joint).

Herein, “bone” means entire bones or bone fragments.

The terms “comprises” and variations thereof do not have a limiting meaning where these terms appear in the description and claims.

As used herein, “a,” “an,” “the,” “at least one,” and “one or more” are used interchangeably. Thus, for example, a composition that comprises “a” polymer can be interpreted to mean that the composition includes “one or more” polymers.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention provides a bone-fusion composition that includes a matrix for bone formation and a growth factor protector and potentiator. Such compositions are particularly useful in methods for fusing vertebrae within the spine of a subject. The compositions can also be used in systems and medical devices that include cage devices (which can be made of materials that are resorbable or nonresorbable in the body of a subject).

The matrix for bone formation preferably includes an osteoconductive carrier such as a calcium phosphate, particularly biphasic calcium phosphate, although other matrices can be used including, for example, collagen, alginate, or combinations thereof. Preferably, the matrix for bone formation is biphasic calcium phosphate. Preferably, such materials are resorbable in the body of a subject.

A particularly desirable biphasic calcium phosphate (BCP), is that which is commercially available under the trade designation BICALPHOS or MASTERGRAFT from Medtronic Sofamor Danek, Memphis, Term. It is a bioresorbable ceramic with a well-defined macroporous structure. The controlled porosity and the presence of interconnection between all the pores can facilitate tissue/cells proliferation inside the material. It is believed that the presence of a controlled specific pore size and the interconnectivity of the pores is that which gives the BCP its osteoconductive properties. Moreover, the bioactive concept of BCP is based on an optimal balance of the more stable phase of HA (calcium hydroxyapatite) and the more soluble TCP (tricalcium phosphate).

The growth factor protector and potentiator is a material (e.g., polymer) that will promote cellular/tissue proliferation, and more particularly will sustain and promote bone fusion in spinal surgery. Preferred materials mimic the properties of heparin toward heparin binding growth factors. In certain embodiments, the growth factor protector and potentiator is a heparin-binding growth factor protector and potentiator (preferably, a dextran derivative).

The growth factor protector and potentiator can be used in a variety of formats. For example, it can be used in solution and administered to the appropriate site via injection. It can be adsorbed onto or covalently bonded to a carrier (e.g., granular material) and/or cage device prior to implantation.

In certain embodiments, the growth factor protector and potentiator is preferably selected from the polymers described in U.S. Pat. App. Pub. Nos. 2001/0021758 or 2001/0023246, or U.S. Pat. No. 6,689,741. This material, which is often referred to as RGTA (ReGeneraTing Agents), is a polymer synthesized from dextran by polysubstitution of the hydroxyl groups with carboxymethyl, benzylamide, and sulfonate groups. RGTAs are functional analogues of heparan sulfate proteoglycans and protect various growth factors from proteolytic degradation, and even enhance their biological activities. RGTA has been shown to promote the healing of defects in tissues such as skin, muscle, intestine, and, especially, bone. Furthermore these molecules induce repair of trephine skull defects in rats, in which no spontaneous repair occurs, and also accelerate the spontaneous healing process observed in long-bone defects.

If desired, the growth factor protector and potentiator can be bound to the matrix for bone formation, either chemically (e.g., covalently) or physically (e.g., adsorbed). If chemically bound, the growth factor protector and potentiator could be coupled to the matrix for bone formation using a wide variety of coupling chemistries. Preferably, for the preferred embodiments of the growth factor protector and potentiator and matrix for bone formation described herein, this can be done using the well-known ester coupling method. Preferably, the ester-coupling agent is a carbodiimide. Carbodiimide is generally utilized as a carboxyl-activating agent for amide bonding with primary amines. Briefly, initially the —OH or —COOH groups of the molecule of interest are reacted with the carbodiimide, which results in the formation of an intermediate group that reacts rapidly with —NH₂ groups. Dicyclohexylcarbodiimide (J. C. Sheehan et al., J. Am. Chem. Soc., 77:1067-1068 (1955)) and 1-ethyl-3-(3-dimethylaminopropyl)carbodiimide (EDC) (J. C. Sheehan et al., J. Org. Chem., 26:2525-2528 (1961)) are commonly used coupling agents. The conditions for such a reaction are well known to one of skill in the art.

In certain embodiments, the growth factor protector and potentiator is a polymer having the general formula (I):
AₐXₓY_{y}
wherein:

A represents a monomer which is substituted with independently selected X and Y groups;

X represents a carboxyl group bonded to monomer A and is contained within a group according to the following formula: —R—COO—R′, in which R is a bond or an aliphatic hydrocarbon chain, optionally branched and/or unsaturated, and which can contain one or more aromatic rings except for benzylamine and benzylamine sulfonate, and R′ represents a hydrogen atom, Y, or a cation;

Y represents a sulfate of sulfonate group bonded to a monomer A and is contained within a group according to one of the following formulas: —R—O—SO₃—R″, —R—N—SO₃—R″, —R—SO₃—R″, in which R is a bond or an aliphatic hydrocarbon chain, optionally branched and/or unsaturated, and which can contain one or more aromatic rings except for benzylamine and benzylamine sulfonate, and R″ represents a hydrogen atom or a cation;

a represents the number of the monomer A such that the mass of said polymers of formula (I) is greater than 5,000 daltons;

x represents a substitution rate of the monomer A by the groups X, which is 20% to 150%; and

y represents a substitution rate of the monomer A by the groups Y, which is 30% to 150%.

In certain embodiments, the growth factor protector and potentiator can include a bound active agent (e.g., amino acids, fatty acids, fatty alcohols, ceramides, or derivatives thereof, and nucleotide addressing sequences). In such embodiments, preferably the growth factor protector and potentiator is a polymer having the general formula (II):
AₐXₓY_{y}Z_{z}
wherein:

A represents a monomer based on glucose which is substituted with independently selected X, Y, and Z groups;

X represents a carboxyl group bonded to monomer A and is contained within a group according to the following formula: —R—COO—R′, in which R is a bond or an aliphatic hydrocarbon chain, optionally branched and/or unsaturated, and which can contain one or more aromatic rings except for benzylamine and benzylamine sulfonate, and R′ represents a hydrogen atom, Y, Z, or a cation;

Y represents a sulfate of sulfonate group bonded to a monomer A and is contained within a group according to one of the following formulas: —R—O—SO₃—R″, —R—N—SO₃—R″, —R—SO₃—R″, in which R is a bond or an aliphatic hydrocarbon chain, optionally branched and/or unsaturated, and which can contain one or more aromatic rings except for benzylamine and benzylamine sulfonate, and R″ represents a hydrogen atom, Z, or a cation;

Z is selected from the group consisting of amino acids, fatty acids, fatty alcohols, ceramides, or derivatives thereof, and nucleotide addressing sequences;

a represents the number of the monomer A such that the mass of said polymers of formula (II) is greater than 5,000 daltons;

x represents a substitution rate of the monomer A by the groups X, which is 20% to 150%;

y represents a substitution rate of the monomer A by the groups Y, which is 30% to 150%; and

z represents the rate of substitution of the monomer A by groups Z, which is 0 to 50%.

Additional details concerning these polymers, particularly their preparation, are described in U.S. Pat. App. Pub. Nos. 2001/0021758 or 2001/0023246, or U.S. Pat. No. 6,689,741. Examples of such materials are RGTA9 and RGTA11 (F. Blanquaert et al., J. Biomed. Mater. Res., 44(1):63-72 (1999); F. Blanquaert et al., Mater. Res. A, 64(3):525-32 (2003); M. L. Colombier et al., Cells Tissues Organs, 164(3):131-40 (1999); and J. Lafont et al., Growth Factors, 16(1):23-38 (1998)).

The bone-fusion system can also include a cage device, particularly an interbody fusion cage for spinal fusion (i.e., an arthrodesis) that is used in combination with the bone-fusion composition. In one embodiment, the matrix (e.g., biphasic calcium phosphate) containing bound or adsorbed growth factor protector and potentiator (e.g., a polymer of the formula AₐXₓY_{y} described above) is placed in the cage, and the latter is inserted between two vertebrae of the area in the spine to be fused. The cage device can be made of a resorbable material or a nonresorbable material. Examples of suitable resorbable materials include, but are not limited to, poly-L,D-lactic acid (PLDLA), poly-L-lactic acid (PLLA), and combinations thereof. Examples of nonresorbable (i.e., non-biodegradable) materials include, but are not limited to, titanium, polyethylethylketone (PEEK), and combinations thereof. The identification and use of cage devices, particularly for spinal fusion, are well-known to one of skill in the art.

In certain embodiments, the bone-fusion composition can further include a growth factor, either in admixture therewith or as part of the growth factor protector and potentiator as described in U.S. Pat. App. Pub. Nos. 2001/0021758 or 2001/0023246, or U.S. Pat. No. 6,689,741. In embodiments of formula II described above, Z is derived from a growth factor. The growth factor is preferably selected from the group consisting of heparin-binding growth factors (e.g., BMP-2 or bone morphogenic protein), basic fibroblast growth factor (bFGF), vascular endothelial growth factor (VEGF), and combinations thereof.

In certain embodiments, the bone-fusion composition can further include stem cells. Suitable stem cells, include for example, bone marrow-derived and adipose tissue-derived stem cells.

The present invention also provides methods for fusing bone. Herein, “bone fusion” refers to permanently joining bone in order to prevent motion, particularly between vertebrae. Herein, “bone” means entire bones or bone fragments.

In one embodiment, such methods of fusing bone involve providing a bone-fusion system of the present invention; placing the composition in contact with bone to be fused; and allowing the bone-fusion composition to harden and fuse the bone.

In one embodiment, such methods of fusing bone involve: providing a bone-fusion system comprising a bone-fusion composition, wherein the bone-fusion composition includes; a growth factor protector and potentiator; and a matrix for bone formation; placing the composition in contact with bone to be fused; and allowing the bone-fusion composition to harden and fuse the bone.

The present invention also provides medical devices that include a cage device and a bone-fusion composition, wherein the bone-fusion composition comprises: a growth factor protector and potentiator; and a matrix for bone formation. Such cage devices are well known to one of skill in the art and could be readily used with a composition of the present invention without undue experimentation.

### EXAMPLE

Objects and advantages of this invention are further illustrated by the following example, but the particular materials and amounts thereof recited in this example, as well as other conditions and details, should not be construed to unduly limit this invention.

### Evaluation of the Performance And Local Tolerance of RGTA in a Bone Lumbar Fusion Model in New Zealand Rabbits

Introduction

The purpose of this study was to evaluate the performance and local tolerance of a bone factor RGTA mixed with a bone substitute (BCP) implanted for 6 weeks to induce bone lumbar fusion in 53 New Zealand rabbits. This test treatment was compared to five other treatment variations: BCP (3 cubic centimeters (cm³)) alone; autologous bone alone used at a volume of 3 cm³ or 1.5 cm³; autologous bone (1.5 cm³) mixed with BCP (1.5 cm³); and autologous bone (1.5 cm³) mixed with BPC (3 cm³) and RGTA.

The radiographic results showed that the BCP (3 cm³) with RGTA induced constantly an increase in bone fusion parameters. Two and four weeks after implantation, the bone fusion rate of the test treatment was superior to the other treatments except for the autologous bone alone (3 cm³) that was constantly associated with the best fusion rate. At week 6, the test treatments with 3 cm³ autologous bone alone and autologous bone (1.5 cm³) mixed with BCP (1.5 cm³) gave the best bone lumbar fusion compared to the other treatments. Under manual palpation no difference between treatments was noticed. No signs of local intolerance were macroscopically observed with the test treatment.

Some inflammation was observed microscopically in most groups but was not considered to be directly related to the treatment. The histological analysis showed that the test article mixed with BCP induced better fusion parameters compared to the five other groups using a modified Emory score. Even though a small sample size was used in this study, the observed trends following the modified Emory score were considered to be of biological significance.

Materials and Methods

Materials

Test treatment: BCP combined with RGTA.

BICALPHOS (BCP) from Medtronic Sofamor Danek is a synthetic bone substitute with a well-defined macroporous structure (approximately 80% porosity, pores of 400-600 nanometer (nm) diameters with inter-connections of 120-150 micron diameters). Sterile BCP granules were used in this study.

RGTA from Regentech SAS (Paris, France) is a heparan-like polymer, synthesized from dextran by a controlled sequential substitution of its glucose units, as described in U.S. Pat. No. 6,689,741, Example 2.

Positive control: Rabbits were implanted with autologous bone under a volume of 3 cm³ per site, obtained from rabbit iliac crest.

Material Preparation

RGTA: a solution of 100 micrograms per milliliter (μg/mL) RGTA was prepared under sterile condition in a 0.9% NaCl solution.

BCP: BCP granules were transferred into a 12 mL sterile tube by the Sponsor and the desired amount (3 cm³ or 1.5 cm³) were measured. Prior to implantation, 5 mL of 0.9% NaCl was added to the BCP granules and mixed for 30 minutes.

Autologous bone: after fascial incisions over the iliac crest, autologous bone chips were harvested from the corticocancellous bone of the iliac crests. The harvested bone was transferred into a sterile bowl and broken down into homogeneously small chips. The bone chips were then transferred into a 12 mL tube. 1.5 cm³ or 3 cm³ were then implanted into the corresponding animals.

Autologous bone+BCP: the autologous bone was prepared as described. The determined amount of autologous bone (1.5 cm³) was mixed with 1.5 cm³ of BCP granules and implanted.

BCP+RGTA: 3 cm³ of BCP granules were mixed with 5 mL of a filtered solution of 100 μg/mL RGTA and shaken for 30 minutes prior to implantation.

Autologous bone+BCP+RGTA: the autologous bone was prepared as described. 1.5 cm³ of autologous bone were mixed with 1.5 cm³ of BCP containing 2.5 mL of a filtered 100 μg/mL solution of RGTA.

Study Design

| | | | Observation Period | |
|---|---|---|---|---|
| | | Number | (weeks) | |
| Group | Treatment | of animals | X-Ray | Sacrifice |
| 1 | Autologous bone (3 cm³) | 4 | 0-2-4-6 | 6 |
| | (positive control) | | | |
| 2 | Autologous bone (1.5 cm³) | 5 | 0-2-4-6 | 6 |
| 3 | BCP (3 cm³) | 5 | 0-2-4-6 | 6 |
| 4 | Autologous bone (1.5 cm³) + | 5 | 0-2-4-6 | 6 |
| | BCP (1.5 cm³) | | | |
| 5 | Autologous bone (1.5 cm³) + | 4 | 0-2-4-6 | 6 |
| | BCP (1.5 cm³) + RGTA | | | |
| 6 | BCP (3 cm³) + RGTA (test | 5 | 0-2-4-6 | 6 |
| | treatment) | | | |

Animal Anesthesia

Each animal was anesthetized with 1 mL xylazine hydrochloride commercially available under the trade designation ROMPUN 2%, BAYER AG, (Germany) and 1 mL ketamine (commercially available under the trade designation IMALGENE 500, MERIAL, France) by intramuscular route. When the animal was placed on the operating table, a solution of xylazine hydrochloride (1 mg/mL) and ketamine (25 mg/mL) in ringer lactate was continuously infused by intravenous route. Each animal was ventilated using a mask.

Once anesthetized, the surgical site of the animals was clipped free or furs scrubbed with a germicidal soap (commercially available under the trade designation VETEDINE, VETOQUINOL, France) and disinfected with povidone iodine (commercially available under the trade designation VETEDINE solution, VETOQUINOL, France). During the surgical procedure, the animal received warm intravenously (I.V.) fluids to prevent dehydratation and help maintain normal body temperature. The reflexes, body temperature and heart rate were also monitored regularly.

Surgery

The surgical procedure was performed under standard aseptic techniques. The L5-L6 vertebral level was estimated by palpation of the iliac crests. A dorsal midline skin incision was made through the skin and two paramedian fascial incisions were performed through the lumbodorsal fascia. The intermuscular plane between the multifidus and longissimus muscles was separated to expose the transverse processes of L5, L6 and the intertransverse membrane. The transverse processes were decorticated using a surgical drilling tool and two identical defects were created symmetrically on each transverse processus.

The prepared materials were placed without excessive compression between the transverse processes in the paraspinal bed on each side of the spine. When all implants were in place, the fascial incisions were closed with absorbable sutures and the skin incision was closed using metallic staples.

Radiographic Evaluation

Postero-anterior radiographs of the L5-L6 lumbar spine were obtained under general anesthesia immediately after surgery, after two, and after four weeks post implantation as well as at sacrifice. The radiographs were then analyzed and the level of fusion was graded using a semi-quantitative grading scale: 0: absence; 1: slight; 2: moderate; 3: marked; 4: complete.

Sacrifice

Animals were sacrificed by lethal injection of barbiturate (available under the trade designation DOLETHAL^{ND}, VETOQUINOL, France) after a 6 weeks observation period.

Manual Palpation

At sacrifice, the lumbar spines were manually palpated at the level of the treated motion segment and at the levels of adjacent motion segments proximally and distally. Each motion segment was graded as solid or not solid (if any motion was present).

Histopathological Samples Preparation

After complete bone fixation into 10% buffered formalin solution, the lumbar specimens were electro-decalcified. The samples were dehydrated in alcohol baths of increasing concentrations and embedded in paraffin blocks. Three parasagittal (longitudinal) sections of 5 μm were cut using a microtom (MICROM, France) in each transverse processes site and through the vertebra arch. Two of the sections were stained with hematoxylin, eosin, and saffron. The remaining section was stained with a Masson trichrome. The Emory score or grading scale was used in this study to evaluate the different treatments and treatment sites. The Emory grading scale is an established histological scoring scale based upon a 0 to 7 score of fibrous tissue, fibrocartilage, and bone content of the fusion mass. This scale was modified in order to adequately take into account the different properties of the test article and control articles evaluated in this study.

Results and Discussion

Radiographic Evaluation

At week 2 and 4 after implantation, the bone fusion of the test treatment was superior to the other treatments except for the autologous bone group (3 cm³) which was constantly associated with the best fusion. A high volume (3 cm³) of autologous bone was constantly associated with better fusion than a low volume (1.5 cm³) of autologous bone at 2, 4 and 6 weeks.

At week 6 post-implantation, the test treatment [BCP (3 cm³)+RGTA; group 6] provided a good level of fusion. The highest level of fusion was obtained by the positive control treatment [autologous bone (3 cm³); group 1] and the autologous bone (1.5 cm³) mixed with BCP (1.5 cm³); group 4. BCP alone showed limited performances, similar to the low volume (1.5 cm³) of autologous bone group.

In summary, six weeks after implantation, the following lumbar fusion grading was obtained:

Autologous bone (3 cm³)=Autologous bone (1.5 cm³)+BCP (1.5 cm³)>BCP (3 cm³)+RGTA>Autologous bone (1.5 cm³)+BCP (1.5 cm³)+RGTA>Autologous bone (1.5 cm³)=BCP (3 Cm³).

These radiographic results indicate that two and four weeks after implantation, the bone fusion rate of the test treatment was superior to the other treatments except for the autologous bone treatment (3 cm³) that was constantly associated with the best fusion rate. At week 6, the test treatment with 3 cm³ autologous bone treatment and autologous bone (1.5 cm³) mixed with BCP (1.5 cm³) gave the best bone lumbar fusion compared to the other treatments. Under manual palpation no difference between treatments was noticed. No signs of local intolerance were macroscopically observed.

Under X-ray, it appears that test treatment (BCP (3 cm³)+RGTA) showed a faster fusion than the other treatments, except the 3 cm³ autologous bone treatment.

Manual Palpation Evaluation

A slight movement of the lumbar spines was observed in each group but no biologically significant difference between groups was observed.

Histological Analysis

Group 1-Autologous bone (3 cm³): a complete fusion, where bone spanned the defect area was not observed for this treatment group. Osteoconduction was very evident in all the sites. The osteoconduction in this study represented an extension of the periosteal reaction in the area of the vertebral arch. The amount of osteoconduction correlated to the amount of periosteal reaction. In most cases the bone chips from the autologous graft did not consistently bridge the implant area. Also in most implant sites the bone chips were spread apart limiting the effectiveness of the autologous bone graft in fusing the area by limiting osteoconduction. The size and distribution of the bone chips were heterogenous and often found embedded into a fibroconnective tissue. Large bone chips were present in some of the vertebral arch and transverse processes sites. These large chips conducted more new bone than accumulations of smaller bone chips and were responsible for the greater amount of new bone present within some of the implant sites. Inflammation was located peripheral to the implant site in one animal. This inflammation did not extend to include the bone graft, but was limited to the soft tissue. The overall performance of this treatment in fusing the adjacent vertebrae was considered poor based upon the microscopic findings in the area of the vertebral arch and was considered moderate in the area of the transverse processes.

Group 2-Autologous bone (1.5 cm³): a complete fusion, where bone spanned the defect area was not observed for this treatment group. The periosteal reaction extended into the implant sites and lead to Modified Emory scores of 6 for four of the ten implant sites for this treatment group around the vertebral arch. As with the previous group, the osteoconduction correlated to the amount of periosteal reaction present. Less newly formed bone was observed between the transverse processes. The placement, space between the bone chips, and size of the bone chips were slightly more consistent in the implant area for this treatment group. As for the previous group, the amount of fibrous tissue was greater than newly formed bone. Inflammation was located peripheral to the implant site in one animal. This inflammation did not extend to include the bone graft, but was limited to the soft tissue. The overall performance of this treatment in fusing the adjacent vertebrae was considered fair. The results from this treatment group around the vertebral arch were slightly better than the first treatment group where more of the autologous bone graft was used. This was due to the placement of the graft material, the amount of periosteal reaction induced, and other factors that affect the variability in this study. There was less newly formed bone between the transverse processes at this level and, the results from the two groups (1.5 cm³ and 3 cm³) were rather comparable.

Group 3-BCP (3 cm³): the BCP consisted of an almost translucent granular material (decalcified material) with consistent large round open (pore-like) areas. The granular matrix had distinct edges and somewhat regular shape. Some of the granular matrix was interconnected whereas in other areas individual pieces of the matrix were present. The granular material was better distributed in the implanted sites compared to the 2 previous sites. Near the vertebrae arch and between the transverse processes, the tissue reaction extended into the round spaces of the matrix to form discrete bony pearls. In other areas fibrocartilage was within the round spaces of the matrix. In most sites the matrix spanned the entire implant area but the density was frequently noted to be decreased in the central areas of the implant site. The implanted material demonstrated very good osteoconductive properties. The same number of vertebral arch sites scored 6 for this treatment as were observed for treatment 2; however, the remaining sites scored slightly better than the remaining sites for treatment group 2. One site was scored 6 between the transverse processes and many of the sites in this treatment group had new bone extending throughout the length of the implant area. The central area of the implant sites (away and between the transverse processes) mostly contained fibrocartilage and fibrous tissue along with a lesser amount of new bone. The fibrous tissue, fibrocartilage and newly formed bone tissue were present in approximative equal amount. Neovessels seemed to increase with area of marked bone ingrowth. This treatment performed better than the previous two treatments.

Group 4-Autologous bone (1.5 cm³)+BCP (1.5 cm³): both components of this test article were evident. The BCP and autologous bone graft were somewhat mixed. The amount of new bone present was associated more with the BCP component than the autologous bone graft. The overall performance of this treatment was similar to the BCP by itself (Group 3). The central areas of the implant typically contained fibrous tissue and fibrocartilage. The test article for one site was infiltrated by large numbers of inflammatory cells.

Group 5-Autologous bone (1.5 cm³)+BCP (1.5 cm³)+RGTA: four of the eight-implant sites were associated with large accumulations of inflammatory cells. This affected the performance of the treatment. The inflammation involved the soft tissue and penetrated into the implant site but the inflammation did not involve the entire implant site. With no accumulations of inflammatory cells observed in the remaining animals, the inflammation observed was most likely not directly associated with the test article. The BCP and the bone chips from the autologous graft were not well distributed throughout the implant site in this treatment group. Very little of the test article components were noted within the central areas of the implant sites. Even with these deficiencies and secondary problems such as the inflammation, this treatment performed similar to the 1.5 cm³ of autologous graft treatment (treatment 2) and better than the 3.0 cm³ of autologous graft (treatment 1) along the vertebral arch as well as between the transverse processes.

Group 6-BCP (3 cm³)+RGTA: this treatment was more consistent between the implant sites than what was observed for the previous treatments. Even though only two of nine vertebral arch sites scored 6, six of the remaining seven vertebral arch sites scored 5 and two of the transverse processes were scored 6. This event never occurred in the other treatments. The central areas of the defects contained fibrous tissue and fibrocartilage; however, subjectively there was more bone associated with the implant sites as a whole. In some areas the bone density appeared elevated. Neovessels seemed to increase with area of marked bone in-growth and were almost comparable with the BCP Group. One animal was associated with an inflammatory response. The etiology of the inflammatory response was not clearly determined, but the inflammation did not involve the entire implanted test article.

In conclusion it seems that the treatment that consisted of 3.0 cm³ of BCP and RGTA (group 6) performed the best and the most consistent. The 3.0 cm³ of BCP (group 3) and the 1.5 cm³ BCP and 1.5 cm³ of autologous graft (group 4) treatments performed well. The 1.5 cm³ and 3 cm³ of autologous graft groups performed similarly, but not as well as the previously mentioned treatment groups. The performance of the group 1.5 cm³ of BCP, 1.5 cm³ of autologous graft+RGTA (group 5) was ranked between the groups 3, 4 and the group 6. The overall performance of autologous bone (3 cm³) was considered poor based upon the microscopic findings. The 1.5 cm³ of BCP, 1.5 cm³ of autologous graft and RGTA treatment sites were complicated by inflammation in 50% of the treated sites. The inflammation observed in this study (including in group 5) was not considered to be directly related to the treatment received. This was made evident by the primary location of the inflammation to the periphery of the implant areas, as well as the observation that the entire implanted test article was not affected. Even though a small sample size was used in this study, the observed trends in the Modified Emory score were considered to be of biological significance.

### CONCLUSION

Two and four weeks after implantation, the bone fusion rate of the test treatment was superior to the other groups except for the autologous bone treatment (3 cm³) who was constantly associated with the best fusion rate. At week 6, the test treatment with 3 cm³ autologous bone alone and autologous bone (1.5 cm³) mixed with BCP (1.5 cm³) gave the best bone lumbar fusion compared to the other treatments. Under manual palpation no difference between treatments was noticed. No signs of local intolerance were macroscopically observed with the test treatment.

Some inflammation was observed microscopically in most groups but was not considered to be directly related to the treatment. The histological analysis showed that RGTA adsorbed on BCP induced better fusion parameters compared to the five other groups using a modified Emory score. Even though a small sample size was used in this study, the observed trends following the modified Emory score were considered to be of biological significance.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A bone-fusion system comprising a bone-fusion composition, wherein the bone-fusion composition comprises biphasic calcium phosphate, and a polymer having the general formula (I):
AₐXₓY_{y}
wherein:
A represents a monomer which is substituted with independently selected X and Y groups;
X represents a carboxyl group bonded to monomer A and is contained within a group according to the following formula: —R—COO—R′, in which R is a bond or an aliphatic hydrocarbon chain, optionally branched and/or unsaturated, and which can contain one or more aromatic rings except for benzylamine and benzylamine sulfonate, and R′ represents a hydrogen atom, Y, or a cation;
Y represents a sulfate of sulfonate group bonded to a monomer A and is contained within a group according to one of the following formulas: —R—O—SO₃—R″, —R—N—SO₃—R″, —R—SO₃—R″, in which R is a bond or an aliphatic hydrocarbon chain, optionally branched and/or unsaturated, and which can contain one or more aromatic rings except for benzylamine and benzylamine sulfonate, and R″ represents a hydrogen atom or a cation;
a represents the number of the monomer A such that the mass of said polymers of formula (I) is greater than 5,000 daltons;
x represents a substitution rate of the monomer A by the groups X, which is 20% to 150%; and
y represents a substitution rate of the monomer A by the groups Y, which is 30% to 150%.

2. The bone-fusion system of claim 1 further comprising a cage device for spinal fusion.

3. The bone-fusion system of claim 2 wherein the cage device comprises a resorbable material.

4. The bone-fusion system of claim 3 wherein the resorbable cage material comprises poly-L,D-lactic acid, poly-L-lactic acid, or combinations thereof.

5. The bone-fusion system of claim 2 wherein the cage device comprises a nonresorbable material.

6. The bone-fusion system of claim 5 wherein the nonresorbable cage material comprises titanium, polyethylethylketone, or combinations thereof.

7. The bone-fusion system of claim 1 wherein the biphasic calcium phosphate and polymer are covalently bonded using an ester coupling agent.

8. The bone-fusion system of claim 7 wherein the ester coupling agent is a carbodiimide.

9. The bone-fusion system of claim 1 wherein the bone-fusion composition further comprises a growth factor.

10. The bone-fusion system of claim 9 wherein the growth factor is selected from the group consisting of heparin-binding growth factors, basic fibroblast growth factor, vascular endothelial growth factor, and combinations thereof.

11. The bone-fusion system of claim 1 wherein the bone-fusion composition further comprises stem cells.

12. A bone-fusion system comprising a bone-fusion composition, wherein the bone-fusion composition comprises biphasic calcium phosphate, and a polymer having the general formula (II):
AₐXₓY_{y}Z_{z}
wherein:
A represents a monomer based on glucose which is substituted with independently selected X, Y, and Z groups;
X represents a carboxyl group bonded to monomer A and is contained within a group according to the following formula: —R—COO—R′, in which R is a bond or an aliphatic hydrocarbon chain, optionally branched and/or unsaturated, and which can contain one or more aromatic rings except for benzylamine and benzylamine sulfonate, and R′ represents a hydrogen atom, Y, Z, or a cation;
Y represents a sulfate of sulfonate group bonded to a monomer A and is contained within a group according to one of the following formulas: —R—O—SO₃—R″, —R—N—SO₃—R″, —R—SO₃—R″, in which R is a bond or an aliphatic hydrocarbon chain, optionally branched and/or unsaturated, and which can contain one or more aromatic rings except for benzylamine and benzylamine sulfonate, and R″ represents a hydrogen atom, Z, or a cation;
Z is selected from the group consisting of amino acids, fatty acids, fatty alcohols, ceramides, or derivatives thereof, and nucleotide addressing sequences;
a represents the number of the monomer A such that the mass of said polymers of formula (II) is greater than 5,000 daltons;
x represents a substitution rate of the monomer A by the groups X, which is 20% to 150%;
y represents a substitution rate of the monomer A by the groups Y, which is 30% to 150%; and
z represents the rate of substitution of the monomer A by groups Z, which is 0 to 50%.

13. The bone-fusion system of claim 12 further comprising a cage device for spinal fusion.

14. The bone-fusion system of claim 13 wherein the cage device comprises a resorbable material.

15. The bone-fusion system of claim 14 wherein the resorbable cage material comprises poly-L,D-lactic acid, poly-L-lactic acid, or combinations thereof.

16. The bone-fusion system of claim 13 wherein the cage device comprises a nonresorbable material.

17. The bone-fusion system of claim 16 wherein the nonresorbable cage material comprises titanium, polyethylethylketone, or combinations thereof.

18. The bone-fusion system of claim 12 wherein the biphasic calcium phosphate and polymer are covalently bonded using an ester coupling agent.

19. The bone-fusion system of claim 18 wherein the ester coupling agent is a carbodiimide.

20. The bone-fusion system of claim 12 wherein Z is derived from a growth factor.

21. The bone-fusion system of claim 20 wherein the growth factor is selected from the group consisting of heparin-binding growth factors, basic fibroblast growth factor, vascular endothelial growth factor, and combinations thereof.

22. The bone-fusion system of claim 12 wherein the bone-fusion composition further comprises stem cells.

23. A medical device comprising the bone-fusion system of claim 1.

24. A medical device comprising the bone-fusion system of claim 12.

25. A method of fusing bone comprising:
providing a bone-fusion system of claim 1 comprising a bone-fusion composition;
placing the composition in contact with bone to be fused; and
allowing the bone-fusion composition to harden and fuse the bone.

26. A method of fusing bone comprising:
providing a bone-fusion system of claim 12 comprising a bone-fusion composition;
placing the composition in contact with bone to be fused; and
allowing the bone-fusion composition to harden and fuse the bone.

27. A method of fusing bone comprising:
providing a bone-fusion system comprising a bone-fusion composition, wherein the bone-fusion composition comprises:
a growth factor protector and potentiator; and
a matrix for bone formation;
placing the composition in contact with bone to be fused; and
allowing the bone-fusion composition to harden and fuse the bone.

28. The method of claim 27 wherein the growth factor protector and potentiator is a heparin-binding growth factor protector and potentiator.

29. The method of claim 28 wherein the heparin-binding growth factor protector and potentiator is a dextran derivative.

30. The method of claim 27 wherein the growth factor protector and potentiator is a polymer having the general formula (I):
AₐXₓY_{y}
wherein:
A represents a monomer which is substituted with independently selected X and Y groups;
X represents a carboxyl group bonded to monomer A and is contained within a group according to the following formula: —R—COO—R′, in which R is a bond or an aliphatic hydrocarbon chain, optionally branched and/or unsaturated, and which can contain one or more aromatic rings except for benzylamine and benzylamine sulfonate, and R′ represents a hydrogen atom, Y, or a cation;
Y represents a sulfate of sulfonate group bonded to a monomer A and is contained within a group according to one of the following formulas: —R—O—SO₃—R″, —R—N—SO₃—R″, —R—SO₃—R″, in which R is a bond or an aliphatic hydrocarbon chain, optionally branched and/or unsaturated, and which can contain one or more aromatic rings except for benzylamine and benzylamine sulfonate, and R″ represents a hydrogen atom or a cation;
a represents the number of the monomer A such that the mass of said polymers of formula (I) is greater than 5,000 daltons;
x represents a substitution rate of the monomer A by the groups X, which is 20% to 150%; and
y represents a substitution rate of the monomer A by the groups Y, which is 30% to 150%.

31. The method of claim 30 wherein the bone-fusion composition comprises a growth factor.

32. The method of claim 31 wherein the growth factor is selected from the group consisting of heparin-binding growth factors, basic fibroblast growth factor, vascular endothelial growth factor, and combinations thereof.

33. The method of claim 27 wherein the growth factor protector and potentiator is a polymer having the general formula (II):
AₐXₓY_{y}Z_{z}
wherein:
A represents a monomer based on glucose which is substituted with independently selected X, Y, and Z groups;
X represents a carboxyl group bonded to monomer A and is contained within a group according to the following formula: —R—COO—R′, in which R is a bond or an aliphatic hydrocarbon chain, optionally branched and/or unsaturated, and which can contain one or more aromatic rings except for benzylamine and benzylamine sulfonate, and R′ represents a hydrogen atom, Y, Z, or a cation;
Y represents a sulfate of sulfonate group bonded to a monomer A and is contained within a group according to one of the following formulas: —R—O—SO₃—R″, —R—N—SO₃—R″, —R—SO₃—R″, in which R is a bond or an aliphatic hydrocarbon chain, optionally branched and/or unsaturated, and which can contain one or more aromatic rings except for benzylamine and benzylamine sulfonate, and R″ represents a hydrogen atom, Z, or a cation;
Z is selected from the group consisting of amino acids, fatty acids, fatty alcohols, ceramides, or derivatives thereof, and nucleotide addressing sequences;
a represents the number of the monomer A such that the mass of said polymers of formula (II) is greater than 5,000 daltons;
x represents a substitution rate of the monomer A by the groups X, which is 20% to 150%;
y represents a substitution rate of the monomer A by the groups Y, which is 30% to 150%; and
z represents the rate of substitution of the monomer A by groups Z, which is 0 to 50%.

34. The method of claim 33 wherein Z is derived from a growth factor.

35. The method of claim 34 wherein the growth factor is selected from the group consisting of heparin-binding growth factors, basic fibroblast growth factor, vascular endothelial growth factor, and combinations thereof.

36. The method of claim 27 wherein the matrix for bone formation comprises an osteoconductive carrier.

37. The method of claim 36 wherein the osteoconductive carrier comprises a calcium phosphate.

38. The method of claim 37 wherein the calcium phosphate is biphasic calcium phosphate.

39. The method of claim 27 wherein the matrix for bone formation comprises collagen, alginate, or combinations thereof.

40. The method of claim 27 wherein the bone-fusion system further comprises a cage device.

41. The method of claim 40 wherein the cage device comprises a resorbable material.

42. The method of claim 41 wherein the resorbable cage material comprises poly-L-lactic acid, poly-L,D-lactic acid, or combinations thereof.

43. The method of claim 40 wherein the cage device comprises a nonresorbable material.

44. The method of claim 43 wherein the nonresorbable cage material comprises titanium, polyetherethylketone, or combinations thereof.

45. The method of claim 27 wherein the growth factor protector and potentiator and the matrix for bone formation are covalently bonded using an ester coupling agent.

46. The method of claim 45 wherein the ester coupling agent is a carbodiimide.

47. The method of claim 27 wherein the bone-fusion composition further comprises stem cells.

48. A medical device comprising a cage device and a bone-fusion composition, wherein the bone-fusion composition comprises:
a growth factor protector and potentiator; and
a matrix for bone formation.

49. The medical device of claim 48 wherein the cage device comprises a resorbable material.

50. The medical device of claim 48 wherein the cage device comprises a resorbable material.
